(19) United States
(12) Patent Application Publication
(10) Pub. No.: US 2014/0148507 A1
(43) Pub. Date: May 29, 2014

(54) PHARMACEUTICAL COMPOSITION AND SOLID GALENIC FORM HAVING A HIGH DRONEDARONE CONTENT, AND METHOD FOR PREPARING SAME

(71) Applicants: Jean Ducassou, Paris (FR); Marie Renouard, Paris (FR)

(72) Inventors: Jean Ducassou, Paris (FR); Marie Renouard, Paris (FR)

(73) Assignee: SANOFI, Paris (FR)

(21) Appl. No.: 14/131,310

(22) PCT Filed: Jul. 6, 2012
(86) PCT No.: PCT/EP2012/063312

§ 371 Date: Jan. 7, 2014

(30) Foreign Application Priority Data
Jul. 7, 2011 (FR) 1156153

(51) Int. Cl. A61K 31/343; A61K 9/20; A61K 9/16

(52) U.S. Cl. CPC A61K 31/343; A61K 9/1694; A61K 9/20
USPC 514/469

(57) ABSTRACT

The present invention relates to pharmaceutical compositions to be used, in a solid galenic form, for oral administration, and primarily including dronedarone and/or at least one of the derivatives thereof, as well as to solid galenic forms manufactured as such from said compositions, preferably in the form of tablets or capsules. The present invention also relates to a method for preparing such solid galenic forms using a hot-melt process.

The present invention relates to pharmaceutical compositions intended for use in solid galenic form for oral administration, comprising primarily dronedarone or at least one derivative thereof, and also to solid galenic forms as such that are manufactured from said compositions, preferably tablets. The present invention likewise concerns a process for preparing such solid galenic forms on the basis of said pharmaceutical compositions by a hot melt route, and also pertains to the therapeutic application of such compositions or of such solid galenic forms.

Dronedarone, or 2-n-butyl-3-[4-(3-di-n-butylaminopropoxy)benzoyl]-5-methylsulfonamidobenzofuran, is represented by the formula (I) below:

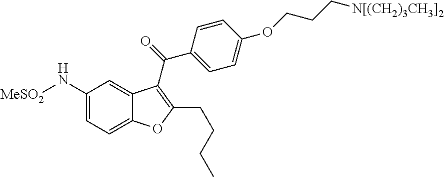

Dronedarone and a method for preparing it are described in patents EP 0 471 609 B1 and U.S. Pat. No. 5,223,510.

Dronedarone blocks potassium, sodium, and calcium channels and also possesses antiadrenergic properties.

Dronedarone is an antiarrhythmic which is effective in maintaining sinus rhythm in patients exhibiting atrial fibrillation or an atrial flutter.

Dronedarone may be administered by means of dosage units containing an effective amount of said active principle, as for example 400 mg of dronedarone for an adult.

For its use as a medicament, dronedarone or a derivative thereof is advantageously formulated in solid galenic forms which are administered orally, such as tablets, for example.

However, certain physical properties of dronedarone present a real challenge in terms of development of formulations suited to the preparation of such solid galenic forms, combining both an effective amount of active principle and a size of said galenic form that is small enough to be easily swallowable.

Specifically, reducing the size of the tablets while maintaining the same amount of active principle involves reducing the amount of the excipients, which poses a number of problems in terms of wettability, disintegration or even total and immediate release of the dronedarone from said tablet.

At the present time, the granulation of dronedarone in the context of the manufacture of a solid galenic form is performed by a wet route. Document EP 1 007 030 B1 describes such a process. The addition of the excipients required for the tableting of this grain and for the assimilation of dronedarone after absorption from a tablet result in a final mass of 650 mg for one unit.

The characteristics of dronedarone form part of the factors which limit the possibilities for increasing its concentration within solid galenic forms. The dronedarone tablet on the market comprises a maximum 65.54% by weight of dronedarone, in the form of dronedarone hydrochloride, relative to the total weight of said tablet.

On the other hand, wet granulation gives rise to unsatisfactory levels of dust emission during the manufacture of the solid galenic forms, following a drying phase which is vital in such a process.

The applicant has discovered, surprisingly, a pharmaceutical composition and a solid galenic form, administrable orally, having a high content of dronedarone and/or of at least one derivative thereof. This capacity to increase the content of dronedarone and/or at least one derivative thereof within solid galenic forms makes it possible to decrease the size of said forms, advantageously tablets, relative to the galenic form currently on the market. The applicant has also discovered a process for hot-melt manufacture of solid galenic forms from such a composition, having the advantage of allowing an increase in the concentration of active principle, advantageously a combined concentration of greater than 80% by weight.

This process via hot-melt granulation has, furthermore, the advantage of overcoming the problems normally encountered in the context of a process via wet granulation. The reason is that this process provides a significantly reduced water content in the galenic form, relative to the use of a conventional process, and the drying step is avoided. The omission of this drying step allows the process to be simplified and made less energy-consuming. The results are a time saving and a cost reduction.

Furthermore, it allows the production of grains based on a composition rich in dronedarone and/or in at least one derivative thereof, these grains being tabletable directly after their production, thereby allowing the implementation of a continuous industrial manufacturing process for dosage units, allowing both a time saving and a cost saving in the industrial production of solid galenic forms, more particularly tablets.

The reduction in the size of the galenic form has a number of advantages:
it allows significant improvement in patient comfort and treatment compliance, since the tablet is more easily ingested by the patient;
it allows the costs of industrial production to be reduced, by virtue in particular of a decrease in the amount of excipients used and a decrease in the cartons and the packaging and therefore in the number of transport pallets.
   The invention provides a pharmaceutical composition comprising:
Up to 92% by weight, advantageously from 81% to 92% by weight, more advantageously between 81% and 86% by weight, of dronedarone and/or of at least one derivative thereof.
7% to 20%, advantageously from 7% to 10% by weight of at least one hot-melt excipient having a melting temperature or a glass transition temperature of greater than or equal to about 35° C. and less than or equal to about 120° C., advantageously less than or equal to about 100° C. and/or greater than or equal to about 50° C., or even greater than or equal to about 35° C. and less than or equal to about 100° C., more advantageously greater than or equal to about 50° C. and less than or equal to about 100° C.;
0% to 20%, advantageously from 0% to 11% by weight of at least one additional excipient other than said hot-melt excipient, selected from disintegrants, lubricants, and flow agents
   the percentages being expressed relative to the total weight of said pharmaceutical composition.
   The invention provides more particularly a pharmaceutical composition comprising:
from 81% to 92% by weight, more advantageously between 81% and 86% by weight, of dronedarone and/or of at least one derivative thereof.
7% to 20%, advantageously from 7% to 10% by weight of at least one hot-melt excipient having a melting temperature or a glass transition temperature of greater than or equal to about 35° C. and less than or equal to about 120° C., advantageously less than or equal to about 100° C. and/or greater than or equal to about 50° C., or even greater than or equal to about 35° C. and less than or equal to about 100° C., more advantageously greater than or equal to about 50° C. and less than or equal to about 100° C.;
0% to 20%, advantageously from 0% to 11% by weight of at least one additional excipient other than said hot-melt excipient, selected from disintegrants, lubricants, and flow agents
   the percentages being expressed relative to the total weight of said pharmaceutical composition.

Dronedarone and the meltable excipient make up the internal phase of the formulation (components used to form the grain). The additional excipients optionally used constitute the external phase of the formulation.

According to another aspect, the invention is likewise directed to a solid galenic form, administrable orally, formed from a pharmaceutical composition according to the invention.

The invention further concerns a process for manufacturing solid galenic forms, comprising at least the following steps, in the indicated order:
a step A of mixing a composition according to the invention;
a step B of granulating said composition using a granulator until a temperature is reached that enables the melting or softening of the hot-melt excipient.

According to one embodiment, the process for manufacturing solid galenic forms comprises the following steps, in the indicated order:
a step A of mixing a composition according to the invention;
a step B of granulating said composition using a granulator until a temperature is reached that enables the melting or softening of the hot-melt excipient.

According to one embodiment, the process is characterized in that it further comprises, following step B, the following steps in the indicated order:
a step C of cooling the grains obtained after the preceding granulation step to room temperature, advantageously between about 20-25° C.;
a step D of calibrating the grains, which involves screening said grains.
   Optionally, the additional excipients forming part of the composition of said invention will be added to the grains during this calibration step. Calibration is advantageously performed through a grille with a mesh size of less than about 2.0 mm;
optionally a step E of mixing said grains, if additional excipients are added during step D;
a step F of shaping the solid galenic forms;
optionally a step G of coating said galenic forms.

The process is characterized in that the solid galenic forms are tablets and/or in that it is a continuous process.

Lastly, the invention relates to a solid galenic form featuring a composition according to the invention, administrable orally and obtainable at the outcome of the process according to the invention, (i) the process advantageously being a continuous process.

The pharmaceutical compositions or the solid galenic forms according to the invention comprise dronedarone or at least one derivative thereof.

In the context of the present invention, the following definitions apply:

Dronedarone Derivatives:

Dronedarone compounds which may also exist in the form of bases or of addition salts with acids. Such addition salts form part of the invention. These salts may be prepared with pharmaceutically acceptable acids, although the salts of other acids useful, for example, for purifying or isolating dronedarone compounds also form part of the invention. The dronedarone derivatives are advantageously addition salts with acids as defined above. More particularly, the derivatives are addition salts with pharmaceutically acceptable acids.
According to one embodiment, the dronedarone derivative is dronedarone hydrochloride.

Excipient: an inactive or inert substance which facilitates the preparation and administration of a medicament;

Disintegrant: an excipient which allows satisfactory breakdown of the galenic form and hence the disintegration of the active principle in the stomach, by increasing the friability and reducing the hardness of the galenic form;

Lubricant: an excipient for facilitating the steps of manufacture of galenic forms, by virtue of its slip-increasing role, i.e., its role in increasing the fluidity of the particles in the pipes of the machines.

Hot-melt excipient: an excipient which softens or becomes fluid under the effect of heat. Such excipients have either a melting temperature: a temperature at which the solid crystal state and the liquid state of the excipient coexist (measured under standard atmospheric pressure of 1 atmosphere), or a glass transition temperature: a temperature below which the molecules forming the excipient have low relative mobility, or the temperature at which the excipient passes from the amorphous solid state into a viscous fluid state.

A pharmaceutical composition or a solid galenic form according to the invention may comprise up to 92% by weight of dronedarone and/or of at least one derivative thereof, advantageously between about 81% and 92% by weight, more advantageously between about 81% and 86% by weight, relative to the total weight of said composition or of said galenic form.

A pharmaceutical composition or a galenic form according to the invention further comprises at least one pharmaceutically acceptable excipient which is hot-meltable at a temperature of greater than or equal to about 35° C., advantageously greater than or equal to about 50° C. and less than or equal to about 120° C., advantageously less than or equal to about 100° C.

The melting temperatures (abbreviated to Tf) or the glass transition temperatures (abbreviated to Tg), where appropriate, that are indicated below are given by way of indication, and are not limitative. Most of them are taken from the “Handbook of Pharmaceutical Excipients”, fifth edition, Pharmaceutical Press.

Hot melt excipients suitable for the invention include citric acid monohydrate (Tf±100° C.), stearic acid (Tf±54-67° C.), palmitic acid (Tf±63-64° C.), lauric acid (Tf±43° C.), myristic acid (Tf±48-55° C.), hydrogenated castor oil (Tf±83-88° C.), hydrogenated plant oil (Tf±57-70° C.), stearyl alcohol (Tf±57-70° C.), cetostearyl alcohol (Tf±49-56° C.), cetyl alcohol (Tf±47-53° C.), vanillin (Tf±76-78° C.), amorphous chlorocresol (Tf±64-67° C.), cetylpyridinium hydrochloride (Tf±80-84° C.), sorbitan monostearate (Tf±43-48° C.), sorbitan monopalmitate (Tf±53-57° C.), xylitol (Tf±92-96° C.), dextrose (Tf±83° C.), ethyl maltol (Tf±89-93° C.), butylated hydroxyanisole (Tf±47° C.), benzalkonium hydrochloride (Tf±40° C.), ascorbyl palmitate (Tf±107-117° C.), erythritol (Tf±119-122° C.), sorbitol (Tf±93-112° C.), shellac (Tf±115-120° C.), maltose (Tf±120-121° C.), sucralose (Tf±130° C.), anhydrous raffinose (Tf±80 118° C.), phenylmercuric borate (Tf±112-113° C.), zinc stearate (Tf±120-122° C.), polyethylene glycol 3000, abbreviated to PEG3000 (Tf±48-54° C.), PEG4000 (Tf±50-58° C.), PEG6000 (Tf±55-63° C.), PEG8000 (Tf±60-63° C.) and PEG20000 (Tf±60-63° C.), polyethylene oxides (Tf±65-70° C.), polyoxyethylene polyoxypropylene copolymers, glyceryl monostearate (Tf±55° C.), glyceryl palmitostearate (Tf±52-55° C.), glyceryl behenate (Tf±65-77° C.), such as, for example, Compritol®888 ATO, ethyl maltol (Tf±89-93° C.), waxes such as, for example, carnauba wax (Tf±80-88° C.), microcrystalline wax (Tf±54-102° C.), white wax or yellow wax (Tf±61-65° C.), beeswax (Tf±61-65° C.), sodium acetate trihydrate (Tf±58° C.), polymethacrylates, in particular Eudragit® E (Tg±48° C.), polyvinyl acetate phthalate (Tg±42.5° C.), carbomers (Tg±100-105° C.), polycarbophils (Tg±100-105° C.), hypromellose acetate succinate (Tg±105° C.), copovidone (Tg±106° C.), and mixtures thereof.

This hot-melt excipient may be advantageously selected from binders, plasticizers, and mixtures thereof. It is advantageously selected from polyoxyethylene polyoxypropylene copolymers, polyethylene glycols abbreviated to PEG, PEG3000 (Tf±48-54° C.), PEG4000 (Tf±50-58° C.), PEG6000 (Tf±55-63° C.), PEG8000 (Tf±60-63° C.) and PEG20000 (Tf±60-63° C.), polyethylene oxides (Tf±65-70° C.), glyceryl monostearate (Tf55° C.), glyceryl palmitostearate (Tf±52-55° C.), glyceryl behenate (Tf±65-77° C.) such as, for example, Compritol®888 ATO, ethyl maltol (Tf±89-93° C.), waxes such as, for example, carnauba wax (Tf±80-88° C.), microcrystalline wax (Tf±54 102° C.), white wax or yellow wax (Tf±61-65° C.), beeswax (Tf±61-65° C.), sodium acetate trihydrate (Tf±58° C.), and mixtures thereof.

With particular advantage the hot-melt excipient or excipients are selected from polyoxyethylene polyoxypropylene copolymers, more advantageously from polyoxyethylene polyoxypropylene copolymers which are hot-meltable at a temperature of between about 50° C. and about 70° C.

Examples include, for example, poloxamer 188 (Tf±52-57° C.), poloxamer 237 (Tf±49° C.), poloxamer 338 (Tf±57° C.), poloxamer 407 (Tf±52-57° C.).

According to one embodiment, the hot-melt excipient is poloxamer 407.

Advantageously, a hot-melt excipient will be selected that has a melting temperature or a glass transition temperature of between about 50° C. and about 120° C., advantageously between about 50° C. and about 100° C. for the manufacture of tablets.

The amount of hot-melt excipient may be from about 7% to 20% by weight, advantageously between about 7% and 10% by weight, relative to the total weight of said composition or of said galenic form.

According to one embodiment, said amount of hot-melt excipient may also be expressed relative to the total weight of dronedarone base equivalent. It will then be between 1% and 20%, particularly between 5% and 15% and even more particularly 10% by weight relative to the total weight of dronedarone base equivalent.

Accordingly, said amount of hot-melt excipient may be defined both relative to the total weight of the composition and relative to the total weight of the dronedarone base equivalent present in the composition.

Base equivalent means the amount corresponding to the amount of dronedarone in base form, in other words unsalified form.

Apart from dronedarone and/or at least one derivative thereof, at least one pharmaceutically acceptable hot-melt excipient, the compositions or the galenic forms according to the invention may include a disintegrant.

This disintegrant may be selected from crosslinked polyvinylpyrrolidone, crosslinked carboxymethylcellulose, alginic acid, sodium alginate, sodium carboxymethylcellulose, microcrystalline cellulose, cellulose powder, sodium croscarmellose, crospovidone, pregelatinized starch, sodium starch glycolate, sodium carboxymethylstarch, starch, and mixtures thereof. The disintegrant advantageously is crospovidone, sodium croscarmellose, or mixtures thereof.

The amount of disintegrant may be between 0% and 10% by weight, advantageously between 3% and 10% by weight, more advantageously between 5% and 10% by weight, relative to the total weight of said composition or said galenic form.

The compositions or the galenic forms according to the invention may further comprise at least one additional excipient other than said hot-melt excipient, which may advantageously have a melting temperature of greater than about 100° C. The additional excipient or excipients is or are selected from customary excipients known to the skilled person, according to the pharmaceutical form and the desired mode of administration. This or these additional excipient or excipients may be selected, for example, from flow agents, lubricants, and disintegrants.

A pharmaceutical composition or a galenic form according to the invention may further comprise at least one lubricant.

This lubricant may advantageously be selected from magnesium stearate, stearic acid, glycerol tribehenate, sodium stearylfumarate, calcium stearate, glyceryl monostearate, glyceryl palmitostearate, sodium lauryl sulfate, zinc stearate, stearic acid, hydrogenated plant oils, polyethylene glycol, sodium benzoate, talc, and mixtures thereof. Advantageously the lubricant is magnesium stearate.

The amount of lubricant may be between 0% and 1% by weight, more preferably between 0% and 0.5% by weight, relative to the total weight of lubricant in said composition or said galenic form.

A pharmaceutical composition or a galenic form according to the invention may further comprise at least one flow agent. The flow agent advantageously is silicone dioxide.

The amount of flow agent may be between 0% and 1% by weight, advantageously between 0% and 0.4% by weight, relative to the total weight of said composition or said galenic form.

According to one embodiment, the composition or the galenic form according to the invention comprises between 81% and 92% by weight of dronedarone hydrochloride, between 7% and 10% by weight of hot-melt excipient, between 5% and 10% by weight of disintegrant, between 0% and 0.5% by weight of lubricant and between 0% and 0.4% by weight of flow agent, relative to the total weight of said composition or of said galenic form.

The pharmaceutical compositions of the present invention are generally formulated in dosage units for daily administration, one or more times a day. In the context of the invention, a dosage unit is a solid galenic form administrable orally, advantageously a tablet.

Accordingly, the dosage unit or solid galenic form, for an adult, may have a mass of 466 mg to 523 mg, and contains 400 mg of dronedarone base equivalent.

They may be plain or coated with various polymers or other appropriate substances.

Starting from the compositions described above, solid galenic forms according to the invention are obtainable by means of the following process.

The process, according to the invention, for manufacturing solid galenic forms, advantageously tablets, takes place continuously.

The process comprises the following steps:
a step A of mixing the ingredients of a composition according to the invention,
a step B of hot granulation using a continuous granulator, by heating said composition to a temperature that enables melting of the hot-melt excipient and the production of an intimate mixture of the ingredients of said composition. This granulation step C is advantageously conducted at a temperature which may be up to about 70° C. to 90° C.

Said steps A and B may take place successively, one after the other, in the indicated order, or at the same time.

and subsequently
a step C of cooling the grains of composition according to the invention, obtained using said granulator, to room temperature, advantageously between about 20-25° C.;
a step D of calibrating, which involves screening the grains obtained in the preceding step D through a grille with a mesh size advantageously of less than 2 mm and/or greater than 1.0 mm, more advantageously of 1.5 mm. The additional excipients of the external phase are optionally added during this step.
Optionally a step E of mixing the grains, when additional excipients are added during step D. This mixing step will allow lubrication of the grains when a lubricant is added, advantageously magnesium stearate.
a step F of shaping galenic forms, advantageously tablets. Tablets are advantageously formed by punch-tableting of grains of composition according to the invention that are obtained at the outcome of step E, where appropriate at the outcome of step F.

Step F is a step of tableting by punching, using a press and punches.

The first step, or step A in said process involves mixing the ingredients of a single composition, manually or mechanically, in mixers, batchwise or continuously.

The continuous granulation step, or step B, may take place in a continuous granulator with corotating screws rotating in a jacketed sheath. This step may be carried out
in a Consigma 25 Collette® granulator with corotating screws, the sheath of which is brought to a temperature of about 80 or 100° C., the screw speed varying from 200 to 400 revolutions per minute.
   Of course, other known equipment and other operating conditions may be used, and form part of the invention (for example, granulation in a fluidized air bed or by compacting).

A third step, also called step. C, then involves cooling, which may be performed, for example, by spreading of the grains obtained in the granulation step on a plate (or by extractive transfer) at about 20-25° C.

A fourth, calibration step D is then conducted, for example, on a Frewitt® TC150 calibrator equipped, for example, with a grille with a mesh size of 1.5 mm. The grille and in particular the mesh size thereof will be chosen according to the desired calibration. The components of the external phase will optionally be added during this step.

Where the formulation contains additional components added as external phase during step D, an optional mixing step, or step E, may take place, by means of a tumbling mixer, for example.

Lastly, the grains undergo galenic shaping, advantageously tableting, by means, for example, of a Korsh® XL 100 rotary press of between 10 and 20 KN with a D-type oblong punch with dimensions of 15.5×8 mm, depending on the desired size and shape.

The pharmaceutical compositions, galenic forms, and manufacturing processes according to the present invention may be marked by the presence of the factors or combinations of factors that are set out below. These factors are, among others:
the amount of hot-melt excipient (for example, of poloxamer), which is at least 7% in the context of the present invention;
the water content of the galenic form; the reason is that the hot melt process provides a significantly reduced water content in the galenic form, relative to the use of a conventional process, as for example a wet granulation process, for the same galenic form. Accordingly, on a majority basis, a water content of 1.5% or less will be found in a tablet, as against a water content of more often between 2% and 2.5% in a tablet manufactured by the known processes, including the wet granulation process;
the size of the tablet relative to its concentration of active principle, but also the concentration of active principle. This is because only the process according to the present invention, to our knowledge, allows dronedarone concentrations of up to 92% to be obtained after tableting, with a single granulating step and without the use of organic solvents. The conventional processes, including, for example, the wet granulation process, necessitate the use of a large amount of excipients in order to enhance wettability of the powder, grain cohesion, and the tabletability of the powder mixture, this amount being directly proportional to the amount of dronedarone employed.

The percentages of the ingredients in the compositions or galenic forms, according to the invention, are selected so as to give a total of 100% by weight, relative to the total weight of said composition or of said galenic form.

### EXAMPLE

The example which follows describes the preparation of pharmaceutical compositions and of galenic forms that are in accordance with the invention. This example is not limiting and merely illustrates the present invention.

Unless otherwise indicated, the percentages of the compounds below are expressed by weight, relative to the total weight of the composition or of the galenic form in which they are located, depending on the specific case.

### 1. Tablet Compositions According to the Invention

By way of example, a pharmaceutical composition 1A and a pharmaceutical composition 1B according to the invention, comprising solely dronedarone hydrochloride as active principle, are indicated illustratively in table 1 below.

The nature of the components in these compositions is indicated in said table, along with the concentration of said components, expressed in % by weight of component relative to the total weight of the composition.

TABLE 1

| Composition | 1A | 1B |
|---|---|---|
| Dronedarone | 91.4% | 81.5% |
| hydrochloride | | |
| Poloxamer 407 | 8.6% | 7.6% |
| Crospovidone | / | 10% |
| Magnesium stearate | / | 0.5% |
| Silicone dioxide | / | 0.4% |

Among these components, there are two used to manufacture the grain—dronedarone hydrochloride and the poloxamer. The other components are additional excipients which improve the processability of the formulation.
Other grains were obtained according to steps A, B and C, described below, using dronedarone hydrochloride as active principle, and other hot-melt excipients. These results are summarized in table 1′ below:

TABLE 1′

| COMPONENT | 1C (in % m/m) | 1D (in % m/m) |
|---|---|---|
| Dronedarone | 90.00 | 90.00 |
| hydrochloride | | |
| PEG 6000 | 10.00 | / |
| (Macrogol 6000) | | |
| Imwitor 491 | / | 10.00 |
| (glycerol | | |
| monostearate > 90%) | | |
| TOTAL (%) | 100 | 100 |

### 2. Continuous Manufacturing Process

Tablets were manufactured using the abovementioned compositions, by the process described below.

A step of mixing (step A) in said process involves mixing in a Turbula® or Servolift® tumbling mixer, for approximately 210 turns, the ingredients of the internal phase of one of the compositions defined above.

This is followed by a step of granulating (step B) using a Consigma® 25 granulator with corotating screws rotating in a jacketed sheath. The parameters are as follows: a powder mixture throughput of 5 to 6 kg/h, a screw speed of 250 to 300 revolutions per minute, a jacket temperature of 80 to 100° C., the temperature of the composition reaching a maximum of 86° C.

A step of cooling (step C) then involves cooling to about 20-25° C. by spreading on a plate or by pneumatic transportation of said grains.

Next, a step of calibrating (step D) is conducted on a Frewitt® TC-150 calibrator by means of a grille with a mesh size of 1.5 mm.

Magnesium stearate, silicone dioxide and crospovidone are optionally added to the grains during step D, where appropriate, with the grains and additional excipients being mixed for approximately 210 turns in a Turbula® or Servolift® tumbling mixer (step E).

The grains, lastly, undergo a tableting step (step F) by means of a Korsh® XL 100 rotary press or a Kilian S100 rotary press at between 10 and 25 KN with an oblong punch 15.5 mm long and 8 mm wide, to form tablets.

Table 2 below collates the characteristics of the tablets 1A and 1B obtained at the outcome of a continuous process with the aid of the compositions described respectively above, and also the characteristics of the reference tablet obtained by wet granulation. For each tablet, table 2 below indicates its total mass and its mass of dronedarone base equivalent (base eq).

TABLE 2

| Tablet | 1A | 1B | Reference |
|---|---|---|---|
| Mass of the | 466 | 523 | 650 |
| tablet (mg) | | | |
| Mass of | 400 | 400 | 400 |
| dronedarone | | | |
| base eq (mg) | | | |

Accordingly, for a given amount of active principle, the compositions according to the invention have a mass and a size reduced relative to the reference tablet.

### 3. Dissolution of the Active Principle of Tablets According to the Invention

3.1. Procedure for Measuring the Dissolution of the Active Principle:

A tablet is placed in a container containing 1000 ml of pH 4.5 phosphate buffer at a temperature of 37° C. The system is stirred at a speed of 75 rpm by means of a blade, and after 30 minutes the amount of dissolved active principle is measured.

3.2. Results

The percentage by weight of dronedarone dissolved for tablet 1B is 83.3%. It is found that the % of dronedarone dissolution is greater than or equal to 80% by weight of dronedarone, relative to the total weight of dronedarone present in the tablet, and this proves to be in agreement with the regulatory recommendations for immediate-release pharmaceutical forms.

### 4. Evaluation of Bioavailability

### 4.1 Protocol

### Treatment and Administration

The dose used is 60 mg/animal irrespective of the period/condition, corresponding to 6 mg/kg (assuming a weight of 10 kg for a dog) and to the dose of 400 mg given to a human (that is, about 6 mg/kg for a human weighing 70 kg).
The administration conditions are as follows:
Period with fasting: the animals are not fed in the evening preceding dosing. Water and routine feed (SSNIFFhdH) are given, respectively, one hour and 4 hours after administration.
Period with feeding: the animals receive 50 g of a high-fat diet (SSNIFF EF Dog FDA Model high fat), 10 minutes before dosing (this diet has an energy value of 100 kcal and is composed of 15% proteins, 25% carbohydrates and 50-60% fat). Water and routine feed for dogs (SSNIFFhdH) are then given, respectively, one hour and 4 hours after administration.
   Pretreatment with pentagastrin is carried out 0.5 hour before dosing. Pentagastrin (6 μg/kg, 0.25 ml/kg) is administered intramuscularly and allows the gastric pH of the animal to be maintained at between 2-3, thus simulating human conditions.
   The treatments are as follows:
   Treatment 1: 60 mg of dronedarone hydrochloride tablet (reference, Multaq®) under fasting conditions, orally
   Treatment 2: 60 mg of dronedarone hydrochloride tablet according to the invention, under fasting conditions, orally
   Treatment 3: 60 mg of dronedarone hydrochloride tablet according to the invention, under feeding conditions with high fat content, orally

### Samples and Analyses

The blood samples are collected in plastic tubes containing lithium heparin as anticoagulant, at the following sampling times: before treatment and 0.5, 1, 2, 3, 4, 6, 8 and 24 hours after administration of each treatment.
The plasma concentration of dronedarone is determined using an exploratory assay method by liquid chromatography coupled to a mass spectrometer (LC-MS/MS). The lower limit of detection with this method for the compounds tested is 0.5 ng/ml.

### Expression of Results

The pharmacokinetic parameters are calculated from the individual concentrations by a noncompartmental analysis using the WinNonLin 5.2.1 software (Pharsight, USA) and using the theoretical sampling times (provided that the actual sampling times do not differ by more than 15% from the theoretical times).
The following pharmacokinetic parameters were measured for each treatment:

Cₘₐₓ (ng/ml): corresponds to the maximum plasma concentration observed,

tₘₐₓ (h): corresponds to the time observed for obtaining the maximum concentration,

AUCₗₐₛₜ: corresponds to the area under the curve or integral of the plasma concentration as a function of the time t calculated by the trapezium method, from tₒ up to the time corresponding to the last quantifiable concentration.

AUC: corresponds to the area under the curve or integral of plasma concentration as a function of time, extrapolated to infinity.

T1/2z; terminal elimination half-life

### Results

Table 3 summarizes the pharmacokinetic parameters following a single oral dose of 60 mg of dronedarone to dogs with controlled gastric pH:

TABLE 3
Pharmacokinetic parameters of dronedarone (mean ± standard
deviation (CV %)) (n = 4 per formulation)

| | Cmax | | AUClast | AUC | |
|---|---|---|---|---|---|
| Treatment | (ng/ml) | tmax (h)* | (ng · h/ml) | (ng · h/ml) | t½z (h) |
| Reference tablet, | 6.67 ± 3.37 | 1.00 | 23.0 ± 12.2 | 25.8 ± 12.9 | 2.53 ± 0.44 |
| with fasting (1) | (51%) | (1.00-2.00) | (53%) | (50%) | (17%) |
| Tablet according | 7.10 ± 5.21 | 1.50 | 26.6 ± 21.3 | 31.1 ± 24.3 | 2.60 ± 0.28 |
| to the invention, | (73%) | (1.00-3.00) | (80%) | (78%) | (11%) |
| with fasting (2) | | | | | |
| Tablet according | 15.4 ± 7.14 | 0.50 | 37.9 + 20.6 | 41.2 ± 22.0 | 2.21 ± 0.24 |
| to the invention, | (46%) | (0.50-1.00) | (54%) | (53%) | (11%) |
| with feeding (3) | | | | | |

*median (min-max)

Table 4 shows the relative bioavailability of each formulation tested, under fasting conditions, by comparison with the reference tablet.

TABLE 4
Relative bioavailability of the dronedarone tablet according
to the invention (%) and 90% confidence interval, observed under
fasting conditions (using the Multaq ® tablet as reference)

| Treatment | Cmax | AUClast | AUC |
|---|---|---|---|
| Tablet according to | 108 (41.0-282) | 120 (41.9-344) | 123 (43.9-347) |
| the invention, | | | |
| fasting, versus | | | |
| reference tablet, | | | |
| fasting | | | |

Under fasting conditions, the Cmax and the AUC observed with the dronedarone hydrochloride tablets according to the invention are similar to those calculated for the reference tablet (Multaq®).
Table 5 shows the meal effect on the bioavailability of the tablet according to the invention.

TABLE 5
ratios of the feeding effect and 90% confidence interval
for the tablet according to the invention

| Treatment | Cmax | AUClast | AUC |
|---|---|---|---|
| With feeding/ | 2.32 (1.32-4.10) | 1.51 (1.01-2.26) | 1.40 (0.97-2.02) |
| with fasting | | | |

There is a positive meal effect tendency when the tablet according to the invention is administered with high-fat food. Specifically, Cₘₐₓ is increased by a factor of 2.3, AUCₗₐₛₜ by a factor of 1.5, and AUC 1.4.

1. A pharmaceutical composition comprising:
from 81% to 92% by weight of dronedarone and/or of at least one derivative thereof;
from 7% to 20% by weight of at least one hot-melt excipient having a melting temperature or a glass transition temperature of greater than or equal to about 35° C. and less than or equal to about 120° C.;
0% to 20% by weight of at least one additional excipient other than said hot-melt excipient, selected from disintegrants, lubricants and flow agents;
the percentages being expressed relative to the total weight of said pharmaceutical composition.

2. The composition as claimed in claim 1, wherein the melting temperature or the glass transition temperature of the hot-melt excipient is less than or equal to about 100° C. and/or is greater than or equal to about 50° C.

3. The composition as claimed in claim 1, wherein the melting temperature or the glass transition temperature of the hot-melt excipient is greater than or equal to about 35° C. and is less than or equal to about 50° C.

4. The composition as claimed in claim 1, wherein the melting temperature or the glass transition temperature of the hot-melt excipient is greater than or equal to about 50° C. and is less than or equal to about 100° C.

5. The composition as claimed in claim 1, wherein the hot-melt excipient or excipients is or are selected from the group consisting of citric acid monohydrate, stearic acid, palmitic acid, lauric acid, myristic acid, hydrogenated castor oil, hydrogenated plant oil, stearyl alcohol, cetostearyl alcohol, cetyl alcohol, vanillin, amorphous chlorocresol, cetylpyridinium hydrochloride, sorbitan monostearate, sorbitan monopalmitate, xylitol, dextrose, ethyl maltol, butylated hydroxyanisole, benzalkonium hydrochloride, ascorbyl palmitate, erythritol, sorbitol, povidone, shellac, maltose, sucralose, anhydrous raffinose, phenylmercuric borate, sorbic acid, hydroxyethylcellulose, magnesium stearate, zinc stearate, polyethylene glycols (abbreviated as PEGs), polyethylene oxides, polyethylenepropylene glycol copolymers glyceryl monostearate, glyceryl palmitostearate, glyceryl behenate, ethyl maltol, waxes sodium acetate trihydrate, polymethacrylates, polyvinyl acetate phthalate, carbomers, polycarbophils, hypromellose phthalate, hypromellose acetate succinate, copovidone, and mixtures thereof.

6. The composition as claimed in claim 1, wherein the hot-melt excipient or excipients is or are selected from the group consisting of citric acid monohydrate, stearic acid, palmitic acid, lauric acid, myristic acid, hydrogenated castor oil, hydrogenated plant oil, stearyl alcohol, cetostearyl alcohol, cetyl alcohol, vanillin, amorphous chlorocresol, cetylpyridinium hydrochloride, sorbitan monostearate, sorbitan monopalmitate, xylitol, dextrose, ethyl maltol, butylated hydroxyanisole, benzalkonium hydrochloride, ascorbyl palmitate, erythritol, sorbitol, povidone, shellac, maltose, sucralose, anhydrous raffinose, phenylmercuric borate, sorbic acid, hydroxyethylcellulose, magnesium stearate, zinc stearate, polyethylene glycols (abbreviated as PEGs), polyethylene oxides, polyethylenepropylene glycol copolymers, glyceryl monostearate, glyceryl palmitostearate, glyceryl behenate, ethyl maltol, waxes sodium acetate trihydrate, and mixtures thereof.

7. The composition as claimed in claim 1, wherein the hot-melt excipient or excipients is or are selected from the group consisting of poloxamers, polyethylene glycols, polyethylene oxides, polyethylenepropylene glycol copolymers, glyceryl monostearate, glyceryl palmitostearate, glyceryl behenate, ethyl maltol, waxes, sodium acetate trihydrate, and mixtures thereof.

8. The composition as claimed in claim 7, wherein the hot-melt excipient or excipients is or are selected from polyoxyethylene polyoxypropylene copolymers.

9. The composition as claimed in claim 1, wherein the hot-melt excipient or excipients is or are selected from binders, plasticizers, and mixtures thereof.

10. The composition as claimed in claim 1, wherein the additional excipient or excipients is or are selected from:
Disintegrant(s) selected from crosslinked polyvinylpyrrolidone, crosslinked carboxymethylcellulose, alginic acid, sodium alginate, sodium carboxymethylcellulose, microcrystalline cellulose, cellulose powder, sodium croscarmellose, crospovidone, pregelatinized starch, sodium starch glycolate, sodium carboxymethylstarch, starch, and mixtures thereof;
Flow agents; and
Lubricants.

11. The composition as claimed in claim 1, wherein said composition comprises at least one component selected from crospovidone, silicone dioxide, magnesium stearate, and mixtures thereof.

12. A solid galenic form, administrable orally, formed from a composition as claimed in claim 1.

13. The solid galenic form as claimed in claim 12, wherein said form is a tablet and/or in that the total weight of said galenic form is from 466 to 523 mg.

14. The galenic form as claimed in claim 12, having a dissolution of greater than or equal to 80% by weight, by weight of dronedarone and/or of at least one derivative thereof, relative to the total weight of dronedarone and/or of dronedarone derivative(s), present in said galenic form, said percentage dissolution being measured after 30 minutes after said galenic form has been placed in a container containing 1000 ml of pH 4.5 phosphate buffer, at a temperature of 37° C. and with stirring at a speed of 75 rpm in a USP 2 apparatus.

15. A process for manufacturing solid galenic forms, comprising at least the following steps, in the indicated order:
a step A of mixing a composition as claimed in claim 1;
a step B of granulating said composition using a granulator until a temperature is reached that enables the melting or softening of the hot-melt excipient.

16. The process as claimed in claim 15, further comprising, following step B, the following steps in the indicated order:
a step C of cooling the grains obtained after the preceding granulation step to room temperature;
a step D of calibrating the grains, which involves screening said grains,
optionally a step E of lubricating the grains by mixing said grains with at least one lubricant;
a step F of shaping the solid galenic forms;
optionally a step G of coating said galenic forms.

17. The process as claimed in claim 15, wherein the solid galenic forms are tablets.

18. The process as claimed in claim 15, wherein said process is a continuous process.

19. A solid galenic form obtainable at the outcome of the process as claimed in claim 15.

20. The galenic form as claimed in claim 19, wherein the process is a continuous process and/or in that the dronedarone is in hydrochloride form.

21. A method for treating and/or preventing pathological syndromes of the cardiovascular system, comprising administrating to a patient at least one galenic form as claimed in claim 12, said galenic form comprising an effective amount of dronedarone and/or of at least one derivative thereof.

22. (canceled)

23. The composition as claimed in claim 5, wherein said polyethylenepropylene glycol copolymers are selected from the group consisting of poloxamer 188, poloxamer 237, poloxamer 338 and poloxamer 407.

24. The composition as claimed in claim 5, wherein said waxes are selected from the group consisting of cannauba wax, microcrystalline wax, white wax, yellow wax and beeswax.

25. The composition as claimed in claim 5, wherein said polymethacrylates is Eudragit® E.

26. The composition as claimed in claim 10, wherein the additional excipient or excipients is or are selected from:
Disintegrants selected from sodium croscarmellose, crospovidone, and mixtures thereof;
Flow agents selected from silicon droxide, magnesium trisilicate, and mixtures thereof; and
Lubricants selected from stearic acid, glycerol tribehenate, sodium stearylfumarate, calcium stearate, glyceryl monostearate, glyceryl palmitostearate, magnesium stearate, sodium lauryl sulfate, zinc stearate, stearic acid, hydrogenated plant oils, polyethylene glycol, sodium benzoate, talc, and mixtures thereof.

27. The process as claimed in claim 16, wherein in step c, said room temperature is between about 20-25° C. and the calibrating of step D is performed through a grille with a mesh size of less than about 2.5 mm.

28. The method according to claim 21, wherein said pathological syndrome of the cardiovascular system is arrhythmia.

29. A method for treating and/or preventing pathological syndromes of the cardiovascular system, comprising administrating to a patient at least one galenic form as claimed in claim 19, said galenic form comprising an effective amount of dronedarone and/or of at least one derivative thereof

30. The method according to claim 29, wherein said pathological syndrome of the cardiovascular system is arrhythmia.
